(19) United States
(12) Patent Application Publication
(10) Pub. No.: US 2023/0187022 A1
(43) Pub. Date: Jun. 15, 2023

(54) DYNAMIC AND MODIFIABLE ENVIRONMENTAL SYSTEM

(71) Applicant: elle Systems Inc., Mountain View, CA (US)

(72) Inventor: Skyyler Siejko, Mountain View, CA (US)

(21) Appl. No.: 18/080,656

(22) Filed: Dec. 13, 2022

Related U.S. Application Data
(60) Provisional application No. 63/289,068, filed on Dec. 13, 2021.

(51) Int. Cl. G16B 20/40; G10H 1/12

(52) U.S. Cl. CPC G16B 20/40; G10H 1/125

(57) ABSTRACT

A computing system that has an application that captures input and develops an integrated environment using biomes with user preferences that are replicated and transferred between different biomes. At least one processor of the computing device is capable of receiving contextual data related to an event and can determine at least one biome for analyzing the received context data and compares a first genetic strand to a second genetic strand related to data associated with the received context data. The processer also determines a third genetic strand, based on the first genetic strand and the second genetic strand, that is a new genetic strand of data different from the first and second genetic strands. The processor also s determines relevancy of the first and second strand and other biomes, and updates the computing system associated with the determined context.

### CROSS REFERENCE TO RELATED APPLICATION

The present application claims priority from U.S. Provisional Application No. 63/289,068 filed on Dec. 13, 2021, which is hereby fully incorporated herein by reference.

### TECHNICAL FIELD

Computing device applications that implement user preferences globally.

### BACKGROUND

Computing systems allow the user to set preferences for a specific application being executed on a computing device or set preferences with the specific device. However, computing systems only have preselected settings and preferences for specific environments.

### BRIEF DESCRIPTION

The current disclosure is a system that runs parallel to an operating system and allows not only the user to use the preferences across an entire computing device and its executed applications, but also across a network with other devices and applications. The current disclosure also has a machine learning aspect where it develops new types of data structures to be used across the network of computing devices and, in some embodiments, may be integrated into each of them.

For the purposes of this disclosure, terms used herein are defined as:
a. DNA and Strand will be used as synonyms until we define a strand structure and the combination of both as a unique set. We will also consider DNA strand to be scoped as local dna and global dna, until further described. Local becoming the Strands within the physical system itself, and Global becoming strands that are put on a defined storage in the cloud or within the physical domain of the technology.
b. Ecosystems, context, playground, and environment are all within a Natural Environmental System (hereinafter referred to as “NES”), and as such, may be thought of within the NES scope or framework.
c. Biome is a context based decentralized network consisting of multiple nodes gaining access to input from users and other devices, not limited to one decentralized hub, but can be extended to and added to by other surrounding biomes to be incorporated to extend context building and deductions. Construction of an established field creates a universal connected language to communicate with other devices using wireless technologies, both analog and digital.
d. Protein may be used in this context as part of creating structures to embed, improve, and operate hardware and software layers alike. Proteins are cultivated and collected to form bigger structures and cluster information in a meaningful way, and is defined as: Any structure where information must be arranged to identify a process or object, which is not restricted to any single or multiple forms.
e. Genes can be defined as a sequence of meaningful symbols and/or data points that may allow for the identification and evolution of a unique set of information. Genes may be used in conjunction with Protein and Biome. Genes are the building blocks for “strand” creation.
f. Organism may be a set or collection of Proteins, which directly translate to an action of desired outcome. Organisms may mutate, replicate, translate, and dictate action performed for the end use case. Organisms are generated as a consequence of the evolution of the Biome.

The invention includes a computing system that has an application that captures input and develops an integrated environment using biomes with user preferences that are replicated and transferred between different biomes. The computing system comprises at least one computing device, wherein at least one processor of the computing device, executes the following, comprising receiving context data related to an event; determine at least one biome for analyzing the received context data, compares a first genetic strand to a second genetic strand related to data associated with the received context data; determines a third genetic strand, based on the first genetic strand and the second genetic strand, that is a new genetic strand of data different from the first and second genetic strands. The system integrates the at least one biome with the third genetic strand and determines relevancy of the first and second strand and other biomes; and updates the computing system associated with the determined relevancy.

The computing system uses proteins that are arranged to identify a process or object, and the computing system creates new proteins. The system uses a DNA structure associated with the user and sends the DNA structure to another system associated with a different user or a different ecosystem of DNA, to become actualized within the user's set context associated with the event. Frequencies are used at the core process to organize and reproduce data from within a core DNA strand, wherein each DNA strand harbors a unique signal. The system further determines commonality between each of the at least one biome and the internal structures and organizes internal structures for maintaining consistency between each respective biome. The biomes are sustainable when integrated within each of the at least one biome, based on genes of the at least one biome, and are not integrated when genes are less likely to be used by the at least one biome. Genetic strands selected determine the growth of the computing system and user preferences of the event. The system may add, by extension, at least one set of musical and digital signal processing rules, bound by transformations. The system further uses harmonics at core processing to organize and reproduce data from within a core DNA strand, and the harmonics are used to construct a new signal. The computing system determines a harmonic key, which acts as a gateway to filter through the signal and find any harmonics within its scale; wherein the computing system may raise the harmonic key by an octave and produce new strands with the same key fundamentals. The computing system may include an angular component, creating vectors from each filtered harmonic and increasing a frequency, based on checking the vector dot product and clearing out any non-zero results, and each strand is constructed with a float value for the frequency, wherein the frequency gets added by a core DNA Process Engine based on determining relevance through a wave resolver. The computing system is based on evolving growth. Completion of a network between multiple noded systems enables growth from between each node of the noded system. The at least one biome of the computing system becomes replicated, cross pollinated and distributed among each biome, and each of the at least one biome communicates with the other biomes.

The computing system for creating the event for the user based on user associated preferences, which are based on the frequency of actions and structured to acknowledge the context between the biome's event frequency, and also recognize variables of the event. Each sum of proteins can create an organism, each organism can create stability by not seeking out the data, but generating the data through a new data type, and not limited by cloud space or infrastructure or physical space. The computing system determines a new type of meta-layer, making an ecosystem of context that evolves and integrates with the computing system in the network and may further allow a user to transfer a DNA strand associated with the user between different contexts, allowing for automation of background and usage specific tasks.

The invention also includes a method for executing an integrated environment application on a computing system, wherein at least one processor of the computing device executes the following steps, comprising receives context data related to an event. The system may determine at least one biome for analyzing the received context data and may compare a first genetic strand to a second genetic strand related to data associated with the received context data. The method of the system may determine a third genetic strand, based on the first genetic strand and the second genetic strand, that is a new genetic strand of data different from the first and second genetic strands; integrates the at least one biome with the third genetic strand. In some embodiments, the method of the system may determine relevancy of the first and second strand and other biomes; and updates the computing system associated with the determined relevancy. The method may further comprise the step of comparing two proteins in a biome and wherein create a third protein different from the compared two proteins in the biome.

### BRIEF DESCRIPTION OF THE DRAWINGS

FIG. 1 illustrates a flow diagram of the computing system determining an event in the system.

FIG. 2 illustrates a flow diagram of the application determining an event.

### DETAILED DESCRIPTION

Various embodiments will be described in detail with reference to the drawings, wherein like reference numerals represent like parts and assemblies throughout the several views. Reference to various embodiments does not limit the scope of the claims attached hereto. Additionally, any examples set forth in this specification are not intended to be limiting and merely set forth some of the many possible embodiments for the appended claims. It is understood that various omissions and substitutions of equivalents are contemplated as circumstances may suggest or render expedient, but these are intended to cover applications or embodiments without departing from the spirit or scope of the claims attached hereto. Also, it is to be understood that the phraseology and terminology used herein are for the purpose of description and should not be regarded as limiting.

The disclosed invention, in one embodiment, may be designed to determine user preferences and change the environment of the system to include the preferences. The system also uses the entire network to determine new structures and processes to create new preferences or data structures for finding new preferences. The system may also include keeping the system updated so that the preferences and parallel system are running with current and updated, and in some instances improved, preferences for the user. The preferences may be used not only in a computing device and integrated with various applications executed on the computing device, but the system may also be used in a network of multiple computing devices. The network may, in some embodiments, use data structures of the system to create new structures and processes for determining user preferences. For the purposes of this disclosure, the example of a calendar or event application are used. However, the disclosure is not limited to an event, but may be used to create a user preferred integrated network system. Therefore, the application and method may use different contexts where the system has a parallel executed process for improving the network environment and integrated with user preferences.

In one embodiment, a computing may be limited to cloud-based platforms and functionality of computing devices. However, the NES may improve this issue, by not seeking out the data, but generating the data through a new data type that may be referred to as “DNA” (or “dna”) not limited by cloud space or infrastructure or physical space. DNA strands are different, in part, which allow for the user's meta-data or actions to be recorded down to a specific set of alleles granted from the genetic construction of its specific usage of the device or context the user or application are in. With emphasis on context may be then record and value the situations not just users are within, but the context that others are within in relation to each user.

Strands may be the culmination of many individual actions becoming pronounced through a genetic sequence expressed naturally by the context any given individual or group DNA is within. The output may be a static data type, however, DNA may constantly evolve within computers systems and computer processes for an anonymous collection of data independent of the user's information. In one embodiment, the system can create a series of active genetic mutations that may become apparent if a series of machines that allow for alternate and stronger genes within DNA to be expressed and populate machines as the natural ecosystem sees fit. The process or natural process of our ecosystem can be defined as any process that would be, or can be used and/or expressed as genetic code (computing code made up of data in a particular group associated with a context), and may be deemed favorable by prediction. In this way, possibilities of one single-use or multi-uses are not limited, but stead, may allow for the natural process of selection (or prediction) and growth between interactions of genes and DNA to frame the understanding, creating a brand-new type of ecosystem with uniquely generated DNA and foundational rules that the systems may follow in a process. The more a user interacts with a system, the more context within the biome can be created and developed, allowing for other gene pools to communicate through an interconnected framework, which can be driven by a natural process such as evolving and mutating with other gene pools, or groups of data. The invention of this disclose may create a new type of meta-layer, making an ecosystem of context that evolves and integrates with the associated machine/user with, and may allow independence to the system to grow naturally and produce reinforce and unique computer actions and schedules.

Genetic learning may be part of the computing system and method. Natural learning from traditional standards allows for single use optimization and solution between input to output, but may not answer the more universal question of how to acclimate the training into an environment. In some instances, segmentation between technology and user may be due to a novice approach within the machine learning paradigm. Entropy can be, in some embodiments, filtered or drained from the hidden layers because those layers may have a unified goal. In one embodiment, an engine may not have a goal, some parallels may be drawn to the natural process of biology (such as the general topics of evolution, natural selection, genetics), and may, in some embodiments, become the default for the system; and integration becomes seamless. The issues arise when we have to discuss the outcome. to say that our system can solve one type of problem relative to another is not wholly correct. In one embodiment, the system is based on not knowing outcomes, and instead follows the development of the system as growth occurs. The completion of a network between multiple node systems, can start to develop growth from and between each respective node. A system or a system of nodes may be referred to as a “biome.” Each biome may become replicated, cross pollinated and distributed among each other biome, just as any other network; the difference is how and when the biomes communicate with each other. training, while important for end-use action, may not deepen the understanding of the biome. Every biome may need to integrate itself with the population of biomes around it. Whether the biome is sustainable depends on the genetic structure of the biome and the population growth (fitness) the growth abides by. Entropy can again remain the important factor in determining the outcome. While entropy grows indefinitely throughout the biome, sampling of the data may not.

A structured aggregation of information can be a determination of how to handle and utilize the entropic development. In this embodiment, the presence of commonality between each biome and the internal structures can be present. Organization of the internal structures (Proteins) may, in some embodiments, contribute to maintaining consistency between each biome. For instance, one biome may create a Protein to represent the amount of times a light should be used throughout the day, while in a completely different biome the same protein exists, but has been defined to acknowledge the context between that biome's lighting frequency. In such an instance, using the biome may impact the result. The difference between those proteins should create a third protein and be utilized to create organisms that have a variable genetic sequence. Each sum of Proteins can create an organism, each organism can create a stability, in come embodiments by freesolves, by not seeking out the data, but generating the data through a new data type we like to call “dna strands,” and may not be limited by cloud space or infrastructure or physical space. DNA strands are different, in part, and allow for the user's meta-data or actions to be recorded down to a specific set of alleles granted from the genetic construction of their specific usage of the device or context. With emphasis on the context, the system can then record and apply a value to the situations, but the context that others, not just the user, are in, relative to each user. The culmination of many individual actions becoming pronounced through a genetic sequence expressed naturally by the context that any given individual or group DNA may be executing on the computing device or generally within associated with the computing device, or the user generally. Instead, in some embodiments, the output being a static data type. DNA may evolve, in some instances continuously, within computers systems and computer processes for an anonymous collection of data independent of the user's information. create a series of active genetic mutations that become apparent if a series of machines that allow for alternate and stronger genes within DNA to be expressed and populate machines as the natural ecosystem sees fit. Natural process of the ecosystem of the computing device can be defined as any process that would be, or can be used and/or expressed as a law of natural science; and is deemed favorable by the laws of predictable nature. The natural process of selection and growth between interactions of genes and DNA to frame the understanding, creating a new type of ecosystem with uniquely generated DNA and foundational rules. The more a user interacts with a system, the more context may occur within that gene pool is developed, allowing for other gene pools to communicate through an interconnected framework which can be driven by a natural process, evolving and mutating with other gene pools. We are creating a new type of meta-layer, making an ecosystem of context that evolves and integrates with the machine/user it is connected to, giving independence to the system to grow naturally and produce reinforce and unique computer actions and schedules. A user can use and own their own DNA structures and transfer them between different contexts, thus allowing a fluid ecosystem of DNA to become actualized within a user's set context. interacting within your own ecosystem, dependent and independent of the device a person is using. allowing for automation of background and usage specific tasks, but the development of a congruent and bettering Environmental System (ES) that run or execute on top, or in parallel, of the Operating System (OS). The total evolution of connected contexts between users, becoming apparent through a natural selection process of keeping and destroying genes that are no longer valid or useful to the user collective. There are two types of Environment Systems: global and local. Local can be classified as any context developed by or for the user within a set system. Global is defined as any context developed by or for the user within a physical space or location, based with or without other user's context(s).

A developer can be defined as any individual who adds or constructs the starting of a context using or manipulating DNA strands. By extension, a developer can build on a framework of evolution controlled by a simple set of musical and digital signal processing rules, bound by transformations within wave mechanical formulation (see MECHANICS). In some embodiments, “Harmonics” can be used at the core process to organize and reproduce data from within a core DNA strand. Each strand can then harbor a unique signal processed by an FFT algorithm, and in some instances, compose a set of unique reproducible signals from a repeated input. Once the signal is present, Harmonics can be added to the FFT in order to construct a new signal. Then, a scale of harmonics can be created and utilized as a map to get back variations of similar inputs. The system creates a complex signal with a fundamental harmonic key. The key can then act as a gateway to filter through the signal and find any harmonics within its scale. We can also take that signal, raise it by an Octave and produce new strands with the same key fundamentals. The properties can then relate the densities of each of the two octaves and match DNA and sequence via common harmonics. Here you can add an angular component, creating vectors from each filtered harmonic and increasing its frequency, based on checking the vector Dot Product and clearing out any non-zero results. Each strand is constructed with a float value for the frequency. The frequency gets added to by our core DNA Process Engine by determining relevance through our Wave Resolver. The system then can use harmonics throughout our Wave Resolver and comparison operation, by doing a filter pass through each signal Array of floats and producing poles of each solid resonance harmonic where the harmonic is the maximum at given points of the filtered signal and tending to zero everywhere else. The filter is established by finding the fundamental of the Root strand (originally created strand), extracting each other signal using IFFT, and taking each left-over component frequencies and finding the harmonic maximums from each component recursively. We then can relate those components to each other by taking the probabilistic overview by relating the percent certainty of a possible match.

The system can, in some embodiments, generate data through a new data type we like to call “dna strands” and may not be limited by cloud space or infrastructure or physical space. DNA strands are different, in part, and allow for the user's meta-data or actions to be recorded down to a specific set of alleles granted from the genetic construction of their specific usage of the device or context. With emphasis on the context, the system can then record and value the situations not just users are within, but the context that others are within in relation to each user. In some embodiments, the culmination of many individual actions may become pronounced through a genetic sequence expressed naturally by the context any given individual or group DNA is within. Rather, the output may be a static data type, but DNA is constantly evolving within computers systems and computer processes for an anonymous collection of data independent of the user's information.

In some instances, evolution may create a series of active genetic mutations that become apparent if a series of machines that allow for alternate and stronger genes within DNA to be expressed and populate machines as the natural ecosystem sees fit. Natural process of our ecosystem can be defined as any process that would be, or can be used and/or expressed as a law of natural science, and may be preferred by the laws of predictable nature. The natural process of selection and growth between interactions of genes and DNA to frame the understanding, creating a new type of ecosystem with uniquely generated DNA and foundational rules.

Bluetooth is overcrowded and best utilized when connectivity is a paramount to the technology. In NES there is less importance put on connectivity and more importance with consistency of data capture. When capturing data and sampling correctly the data trends, purity may be increased, and in effect, allow for the development of more consistent strands. The instances where strands are the backbone of the application of technology, the rate at which we create them is less important than the strength of the dna relative to previous sampling. FM frequency range seems to suffice and takes little power to create and capture throughout the biome. The biome extended range may provide the user within the biome the security and sensitivity needed to capture significant and accurate data. The system may treat previous computers and devices as nodes within the biome and add receivers to needed areas, for receiving signals such as an FM signal, and may send bluetooth to device. With this idea of universal language for compliance, a standard for other devices may be implemented.

A data throughput may allow for generational and systematic building, designing, evolving, and mutating through continual sequencing both in a global and local context. The service takes any source input and does a series of ASCII conversions, sending a throughput into the Ocean, to be sequenced into associated wave component parts. This service outputs a DNA-Object, that may become a throughput of data, manipulating the input data into signals for further processing and transforming to appropriate outputs and comparisons. The main engine, which utilizes harmonics and FFT to form DNA output data at this time input, can be constructed to become an event-Object or a DNA strand as classified by the internal algorithm Generally, an engine when related to software may be a program that performs a core or essential function for other programs. It may have a set of rules or analysis to process data. An engine may be used in operating systems, subsystems or application programs to coordinate the overall operation of other programs. In some embodiments, this service only generates and classifies DNA inputs. The system can utilize competing strands from the Evolver and will generate or discriminate between existing local Evolver strands and current strands being momentarily generated (aka for events). This process allows for the DNA to be temporarily stored and sorted by frequency.

The Evolver also incorporates two threshold requirements with its stored DNA. Both thresholds utilize the frequency as a limiter. Threshold global may be used to determine if the local strand should check the database strand or it should remain in the Evolver. Threshold local can be used to decide which strands locally should be added to the local DNA file system, or if the strand is not strong enough or used frequently enough will be eliminated from the Evolver. A strand may be converted into an event object and used to either add to the order, and may eventually pop off to run a system process using the taskmaster, or get added to the tracked time and put into a timeline for future use and system involvement used as a temporary place to keep track of order events and procedures. In some embodiments, the strand may be a momentary snapshot of the current event-object and handles the system load independent of any other service. The strand may drain into a cache if determined to check, relate, and give systematic context to the DNA. The DNA is given the frequency and can also be eliminated during processing. In this instance, each DNA strand may be given a pre-check for validation, and is also read into the data stream.

In some instances, the system may include a service which receives event-objects and generates an executable action on the OS layer, and may receive events directly from the order or time timeline. This can be considered to be accepted and provided permission by the user/machine that the service is running on. A grouping of event-objects may allow for usage of a collection of DNA between each strand. In other instances, an object may be a sequence of multiple DNA contexts such as an input system that ingests the data from the input source and decides whether to eliminate or send to our core engine. Such an engine can be built within and below the OS layer and uses C/C++ to generate consistent and optimized inputs. A health check and redundancy layer may also be included, permitting self-healing properties, using time interval checks and maintaining a local cache within the entire system itself. In this way, the system may provide graceful shutdown and boot-up of services alongside interconnected services of all other processes, and may eventually provide the security layer for local and global strand pathways. The system may resolve this issue by not seeking out the data, but generating the data through a new data type called “dna strands” where the system is not limited by cloud space or infrastructure or physical space.

In one embodiment of the procedure of steps, each service, respectfully, converts a context of information within a set environment, may it be a user action or environmental consequence such as crowd-sourced DNA thresholds being met. At a minimum, both services are acting in the Environmental System, and additional core engine modules may be introduced based on quality and quantity of DNA from within the constraints of the Environmental System to which the input data source is maintaining and receiving. Output DNA then becomes a one way translated language in which the Environmental System can translate, allowing for specific features to be understood and enacted.

Each DNA-object is compared to the subsequent strand DNA within the local dna file and is broken up into component frequencies via Harmonics. The bandwidth of each signal starts with an impulse where a system can be defined with a single maximum and an evenly distributed minimum throughout the signal. Once the signal is established; each DNA frequency is applied using FFT to add the signal and perform a cooley-radix, giving the appropriate output signal to the Wave Resolver, and thus, creating a plausible signal. In some examples, each component frequency may then be transformed and compared to each of the other component frequencies by finding fundamentals of the harmonic and establishing a density function that collapses the probability to an initial maximum, and thus, comparing two signals and matching the fundamentals.

We create a foundation layer on top of the OS that acts as a new type of System within both personal computers and industrial mechanized computer systems. The system creates a NES. Therefore, the evolution of the system remains congruent with the input of the user/target device. In some instances, there may be a strong correlation in device input and creation of DNA. However, DNA is not necessarily input based and can be created from a generalized global database of DNA from various other structural inputs, may it be machine, or the user. There can also be a separation of ecosystems within a company/entity. Modularized to only consider and include creation of strands from within the entity itself, creating a proprietary ecosystem that can be used, sold and demonstrated to other entities. The system may create an open network, by which other entities may connect to in order to derive more complex DNA structures to the ever evolving proprietary and nonproprietary modules. When using this type of system, the framework may allow for multiple machine learning applications to gain access and improve from. Machine learning (“ML”) systems are able to learn, and the system may be the “playground” or an environment constructed of transformation matrices which promotes other systems to learn from each other with a foundational rule set defined by our Genetic Engines Transformational data. ML may interact with the playground, without having a structured understanding of how to use the playground, so ML processes can act as play and activities like “children” interacting with other children through a set of social rules. The system provides both the “social construct” of the playground as well as the activity each child decides to partake in. Entities (or children) then become able to utilize each other's ML structured data into a unified language called strands. The conditions of the “social contract” of the ecosystem/environment govern the global ecosystem and are decentralized into multifaceted ports that can generate unique DNA between two children allowing for new “games” or activities to be played on the ever-evolving playground. In this way, multiple ecosystems may interconnect and geolocalize throughout physical space, allowing for a generation of strands to be cultivated and reproduce within a stable environment.

A blank biome may be a place where species evolve and grow to produce consequences to that ecosystem. The process may include raw user/device data and what comes out may be context that can be used to determine the patterns of an individual/device, a group of individuals/devices, and a system of individuals/devices. In this way, the system may create a natural system of communication/interaction between devices, groups of devices, and groups of users. A timeline and systematic way of making the processor more efficient and functional through limiting and pushing through different processes as the DNA processor sees throughout the lifetime of the device. A timeline and systematic way of making the processor more efficient and functional through limiting and pushing through different processes as the DNA processor sees throughout the lifetime of the device. In this embodiment, a specialized and unique data set may then create and distribute unique operating systems without the need for setup and integration and may create a specific preset for the system based on the user's past actions and even anticipates presets that the user needs. The system may share DNA in such a way that allows for each individual strand to grow and evolve and may allow your computer to learn your actions and habits, and the user's needs and requirements. In some instances, files may be uploaded for others to use as well as download them to change your own system. By downloading, the system may either change the way that the system works entirely or let the biome change the system slowly over time to help smooth out the way the user interacts with the user's computing device. The system may also remember settings input by the user or created by the system. The system can be copied and implemented for other users to create shortcuts, store and proactively search for relevant information, and overall improve the efficiency of use of the computing device.

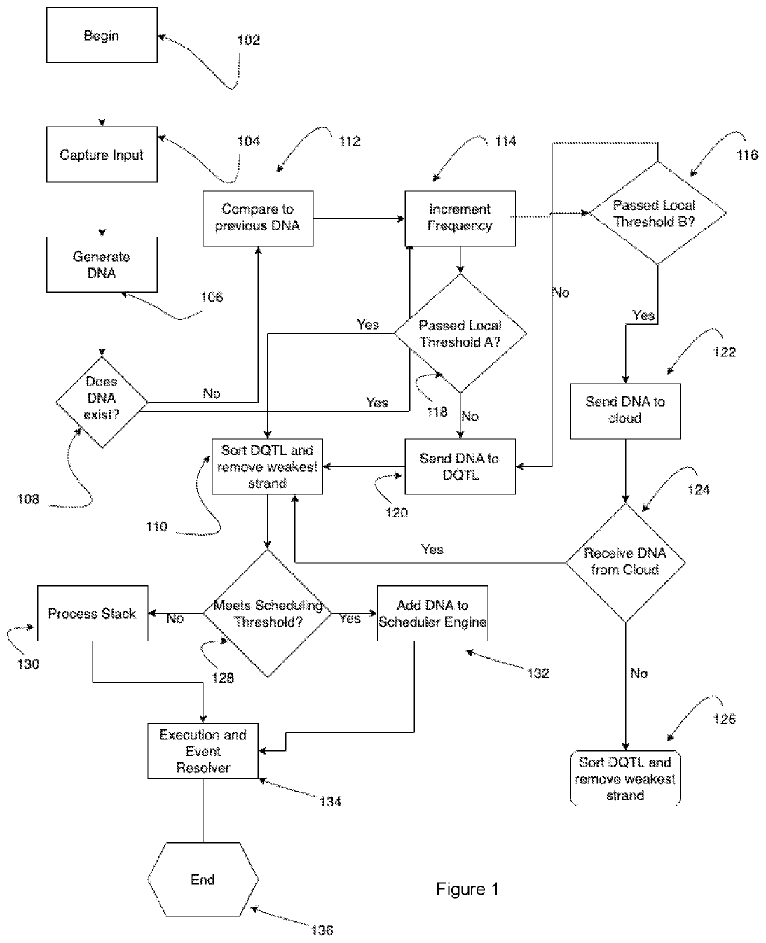

In FIG. 1 (FIG. 1), the computing system flow diagram begin with “Begin” block 102 that may be execution of a computing device by a user (not in the flow diagram). The execution of the computing device may then send data, or the computing device may receive data, in capture input 104. This may also include using the computing device and executing apps such as taking photos, looking up locations, looking up contacts, etc. or any other executable app on the computing device. The computing system analyzed the data captures and executed at the computing device. The system further generates DNA 106, which DNA may be a strand structure and the combination of both as a unique set of data. The data associated with the event and context of the event and computing device usage. Once the DNA is generated 106, the computing system looks at if the DNA exists 108. If DNA (data) exists, then the system will increment the frequency 114. If there is no DNA determined, then the system will compare the data to previous DNA 112. The process may then continue to increment frequency 114 if there is DNA found to exist. If not, then they system begins the process again when the next input is captured 104. After the step of increment frequency 114, the computing system that applied a local threshold to the frequency. In some examples, the frequency may be changed by the system. In some embodiments, the system may apply values, so comparing the resulting determined values to a predetermined threshold. The incremental frequency 114 may then be compared to local threshold A 118. Increment frequency 114 may look at harmonics in some embodiments, as described herein. Harmonics may have values or frequencies that are compared to thresholds. If the data passes the local threshold A 118, then the Dynamic Queue Transport Layer (“DQTL”) may receive the data and can sort the data. In some embodiments, the DQTL may remove the weakest strand 110. If the data does not pass local threshold A 118, then the DNA is sent to the DQTL 120 for sorting. If the data passes local threshold B, then the DNA is sent to a cloud 122. In some embodiments, the received DNA from the cloud 124 may be further be analyzed by the computing system. DNA received by the cloud may then be sent to the sort DQTL 110 and removal of the weakest strand may occur. Removal may be based on a value determined, frequency, contact, relevancy, likelihood of use, etc. If no DNA was received from the cloud 124, then the data is sent to sort DQTL and the weakest strand is removed 126.

As mentioned, regarding the increment frequency 114, the data may be sent to determine if it passes local threshold B 116. If threshold B is not met, then the DNA is sent to DQTL 120.

From sort DQTL 110, the data is then sent to determine if the data, in some embodiments not including the removed weakest strand, meets a scheduling threshold 128. If the data, or data values, does meet the scheduling threshold 128, then it is sent to add DNA to scheduler engine 132. If it does not meet the scheduling threshold 128, then it's sent to process stack 130. In both determinations of meets scheduling threshold 128, the data may then be sent to execution and event resolver 134 to create the event preference and continue to end 136 or to start again. Execution and event resolver 134 may cause the computing device of the user to implement the event preferences. The data sent to the Scheduler Engine, may be stored in the computing system with the new DNA locally or in other biomes of the system, globally for the entire system to use.

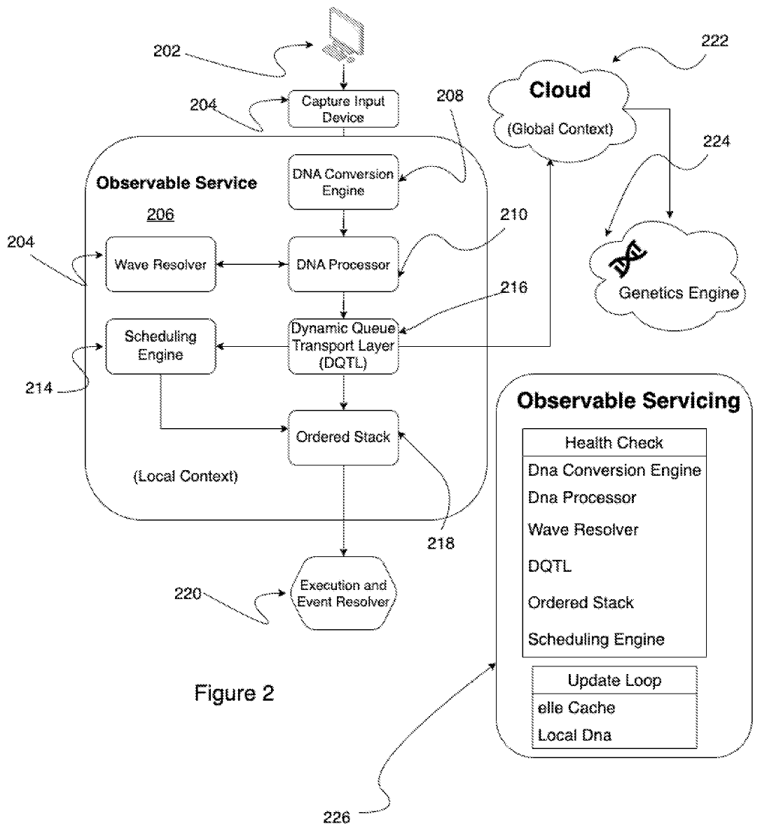

In FIG. 2 (FIG. 2), a data logic flow diagram, the computing device 202 may be used by a user (not illustrated) to execute various applications on a computing device. During the use of computing device 202, input data may be captured by capture input device 204. Captured input data from captured input device 204 may then be sent to observable service 206. In some instances, the captured input data may be received by DNA conversion engine 208, of observable service 206, where the any modification of DNA may occur and then sent to DNA processor 210. The DNA processor 210 then sends any processed data to a wave resolver 212, and may send wave resolve data back to DNA processor 210 for further processing or to go back in the system. DNA processor 210 may then send the processed DNA data to Dynamic Queue Transport Layer (“DQTL”) 216. DQTL 216 is in communication with a cloud 222 which is a global context store for the computing devices connected in the system and communication with the biomes of the network (not illustrated). Cloud 222 may communicate or transfer the data to Genetic Engine 224 for storage of DNA and Genetic rules and data.

Data sent by DQTL 216 may then be sent to scheduling engine 214 for further determination of the event and determined context of the event. DQTL may also receive data from cloud 222 which may include data from other biomes or computing systems in the network in communication with cloud 222. The cloud is updated with genetic engine data received. One embodiment is a new DNA strand created by genetic engine 224 that include genes from the network (via cloud 222) or by comparison of the genetic engine 224. The new DNA strand is sent to cloud 222 to put into the DQTL 218 or into the network of all computing devices to be used for others scheduling event. DQTL 216 may then send the data to ordered stack 218 for putting the data in an organized manner and determined order or stack. Ordered stack also received data from scheduling engine 214, and the received data may also be included in the ordered stack 218 data. Ordered stack 218 then sends the data to execution and event resolver 220. This updates the device according to the ordered stack 218 sent data. The execution and event resolver 220 execute the event preferences on computing device 202 and are stored back in the system for future preference analysis.

Observable Services may include storage of data. Such as cached data and local DNA data. The storage of Observable Servicing 226 may also include Health-Check information. Generally, storage device(s) on a computing device may be configured for short-term storage of information as volatile memory and therefore not retain stored contents if powered off. Examples of volatile memories include random access memories (RAM), dynamic random access memories (DRAM), static random access memories (SRAM), and other forms of volatile memories known in the art. Storage devices may further be configured for long-term storage of information as non-volatile memory space and retain information after power on/off cycles. Examples of non-volatile memories include magnetic hard discs, optical discs, floppy discs, flash memories, or forms of electrically programmable memories (EPROM) or electrically erasable and programmable (EEPROM) memories.

One or more processors may implement functionality and/or execute instructions within the computing device. For example, processor(s) on the computing device may read and execute instructions stored by storage devices that execute the functionality of application and hardware of the computing device. These instructions executed by processors may cause the computing device to store information within storage devices during program execution, such as user DNA data, and/or objects generated by or associated with one or more of applications, such as an event or calendar application and user input data. Processors may execute instructions of preferences to determine a weighted probability for one or more pieces of data in the DNA strand or genes processed.

While embodiments of the invention have been illustrated and described, it will also be apparent that various modifications can be made without departing from the scope of the invention. It is also contemplated that various combinations or sub combinations of the specific features and aspects of the disclosed embodiments can be combined with or substituted for one another in order to form varying modes of the invention. Accordingly, it is not intended that the invention be limited, except as by the appended claims. All references cited within are herein incorporated by reference in their entirety.

What is claimed is:

1. A computing system that has an application that captures input and develops an integrated environment using biomes with user preferences that are replicated and transferred between different biomes; wherein the computing system comprises at least one computing device, wherein at least one processor of the computing device executes the following, comprising:
receives context data related to an event;
determine at least one biome for analyzing the received context data;
compares a first genetic strand to a second genetic strand related to data associated with the received context data;
determines a third genetic strand, based on the first genetic strand and the second genetic strand, that is a new genetic strand of data different from the first and second genetic strands;
integrates the at least one biome with the third genetic strand;
determine relevancy of the first and second strand and other biomes; and
updates the computing system associated with the determined relevancy.

2. The computing system of claim 1, wherein the computing system uses proteins that are arranged to identify a process or object, and the computing system creates new proteins.

3. The computing system of claim 1, wherein the system uses a DNA structure associated with the user and sends the DNA structure to another system associated with a different user or a different ecosystem of DNA, to become actualized within the user's set context associated with the event.

4. The computing system of claim 1, wherein frequencies are be used at the core process to organize and reproduce data from within a core DNA strand, wherein each DNA strand harbors a unique signal.

5. The computing device of claim 1, wherein the system further determines commonality between each of the at least one biome and the internal structures; and organizes internal structures for maintaining consistency between each respective biome.

6. The computing system of claim 1, wherein the biomes are sustainable when integrated within each of the at least one biome, based on genes of the at least one biome, and are not integrated when genes are less likely to be used by the at least one biome.

7. The computing device of claim 1, wherein the genetic strands selected determine the growth of the computing system and user preferences of the event.

8. The computing system of claim 1, wherein the system may add, by extension, at least one set of musical and digital signal processing rules, bound by transformations.

9. The computing system of claim 1, wherein the system further uses harmonics at core processing to organize and reproduce data from within a core DNA strand, and the harmonics are used to construct a new signal.

10. The computing system of claim 1, wherein the computing system determines a harmonic key, which acts as a gateway to filter through the signal and find any harmonics within its scale; wherein the computing system may raise the harmonic key by an octave and produce new strands with the same key fundamentals.

11. The computing system of claim 1, wherein the computing system includes an angular component, creating vectors from each filtered harmonic and increasing a frequency, based on checking the vector dot product and clearing out any non-zero results, and each strand is constructed with a float value for the frequency, wherein the frequency gets added by a core DNA Process Engine based on determining relevance through a wave resolver.

12. The computing system of claim 1, wherein the computing system is based on evolving growth, wherein completion of a network between multiple noded systems enables growth from between each node of the respective noded system.

13. The computing system of claim 1, wherein each of the at least one biome of the computing system becomes replicated, cross pollinated and distributed among each biome, and each of the at least one biome communicates with the other biomes.

14. The computing system of claim 1, wherein the computing system for creating the event for the user based on user associated preferences; wherein the created preferences are based on the frequency of actions and structured to acknowledge the context between the biome's event frequency, and also recognize variables of the event.

15. The computing system of claim 1, wherein each sum of proteins can create an organism, each organism can create stability by not seeking out the data, but generating the data through a new data type, and not limited by cloud space or infrastructure or physical space.

16. The computing system of claim 1, wherein the computing system determines a new type of meta-layer, making an ecosystem of context that evolves and integrates with the computing system in the network.

17. The computing system of claim 1, wherein the computing system further allows a user to transfer a DNA strand associated with the user between different contexts, allowing for automation of background and usage specific tasks.

18. A method for executing an integrated environment application on a computing system of claim 1, wherein at least one processor of the computing device executes the following steps, comprising:
receives context data related to an event;
determine at least one biome for analyzing the received context data;
compares a first genetic strand to a second genetic strand related to data associated with the received context data;
determines a third genetic strand, based on the first genetic strand and the second genetic strand, that is a new genetic strand of data different from the first and second genetic strands;
integrates the at least one biome with the third genetic strand;
determines relevancy of the first and second strand and other biomes; and
updates the computing system associated with the determined relevancy.

19. The method of claim 19, further comprising the step of comparing two proteins in a biome and wherein create a third protein different from the compared two proteins in the biome.
